(19) United States
(12) Patent Application Publication
(10) Pub. No.: US 2008/0300409 A1
(43) Pub. Date: Dec. 4, 2008

(54) Crystalline clopidogrel hydrobromide and processes for preparation thereof

(75) Inventors: Nina Finkelstein, Herzliya (IL); Judith Aronhime, Rehovot (IL)

Correspondence Address: KENYON & KENYON LLP, ONE BROADWAY, NEW YORK, NY, 10004, US

(73) Assignee: Teva Pharmaceuticals USA, Inc. for Barbados

(21) Appl. No.: 12/221,349

(22) Filed: Jul. 31, 2008

Related U.S. Application Data
(62) Division of application No. 11/233,491, filed on Sep. 21, 2005.
(60) Provisional application No. 60/611,995, filed on Sep. 21, 2004.
(60) Provisional application No. 60/615,771, filed on Oct. 4, 2004.

(51) Int. Cl. C07D 471/02

(52) U.S. Cl. 546/114

(57) ABSTRACT

Provided are crystalline forms of clopidogrel hydrobromide and processes for their preparation.

### RELATED APPLICATIONS

This application is the divisional of U.S. patent application Ser. No. 11/233,491, filed on Sep. 21, 2005, which claims the benefit of U.S. provisional application Nos. 60/615,771, filed Oct. 4, 2004 and 60/611,995, filed Sep. 21, 2004, the contents of all of which are incorporated herein.

### FIELD OF THE INVENTION

The present invention relates to the solid state chemistry of clopidogrel hydrobromide.

### BACKGROUND OF THE INVENTION

Atherosclerosis is the buildup of plaque in the wall of the arteries leading to a thickening and a reduction in elasticity of the arteries. Atherosclerosis results from injury to the inside layer of the artery. The injury is caused by common activities and diseases such as high cholesterol, high blood pressure, smoking and infection.

Plaques form on the inner walls of the artery at these sites of injury. The plaques are mainly composed of fatty tissue and smooth muscle cells. The formation of plaque often leads to blood clotting due to platelet aggregation at the site of the injury. This clotting may result in a reduction or elimination of blood flow to vital organs, causing heart attacks or other serious conditions. The plaque may also rupture and send a blood clot through the artery, referred to as an embolus, which if deposited in a smaller blood vessel may completely block blood flow.

Anti-platelet activity is desirable in fighting the often fatal results of atherosclerosis. Clopidogrel is an inhibitor of induced platelet aggregation which acts by inhibiting the binding of adenosine diphosphate to its receptor. Clopidogrel is metabolized by the liver into active form. Its anti-platelet activity is extended in that it stops any platelet activity even up to ten days after administration.

The chemical name of clopidogrel is methyl (+)-(S)-∝-(o-chlorophenyl)-6,7-dihydrothieno[3,2-c]pyridine-5(4H)-acetate. It has the following structure:

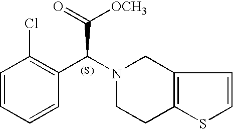

Clopidogrel's platelet inhibiting activity makes it an effective drug for reducing the incidence of ischemic strokes, heart attacks or claudication due to vascular diseases such as atherosclerosis. By inhibiting platelet aggregation, clopidogrel reduces the chance of arterial blockage, thus preventing strokes and heart attacks. U.S. Pat. No. 5,576,328 describes a method of preventing the occurrence of a secondary ischemic event by administration of clopidogrel, and is incorporated herein as a reference.

Recent studies have shown that clopidogrel is more effective in blocking platelet aggregation than aspirin and is much gentler on the gastrointestinal tract. Clopidogrel is more effective than aspirin even at much lower dosage. A dosage of 75 mg of base equivalent has been shown to be more effective than a dosage of 325 mg of aspirin. In addition to being more effective, clopidogrel produces much less gastrointestinal bleeding than aspirin.

Clopidogrel is currently being marketed as PLAVIX® tablets, which contain about 98 mg clopidogrel hydrogensulfate, which is the equivalent of 75 mg clopidogrel base. PLAVIX® is a white to off-white powder that is practically insoluble in water at neutral pH but highly soluble at acidic pH. It dissolves freely in methanol, somewhat in methylene chloride, and poorly in ethyl ether.

U.S. Pat. Nos. 4,847,265; 5,132,435; 6,258,961; 6,215,005 and 6,180,793, which are hereby incorporated by reference in their entirety, describe methods that can be used to prepare clopidogrel hydrogensulfate.

(+)-Clopidogrel, disclosed in U.S. Pat. No. 4,847,265, is an oil, whereas its hydrobromide salt exists as a powder, which can be used for the purification of clopidogrel followed by its conversion to the hydrogensulfate, or it can be used for preparation of pharmaceutical composition. The preparation of (+)-Clopidogrel hydrobromide is described in example 1 of the '265 patent. The product is identified by a melting point of 140° C.

The following publications also relate to solid state chemistry of clopidogrel salts: WO2005/080890 and WO 2005/068471, CZ20050149 and CZ20040061

The present invention relates to the solid state physical properties of the clopidogrel hydrobromide. These properties can be influenced by controlling the conditions under which clopidogrel hydrobromide is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account when developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulation syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. These conformational and orientation factors in turn result in particular intramolecular interactions with adjacent polymorphic form may give rise to distinct spectroscopic properties that may be detectable by powder X-ray diffraction, solid state ¹³C NMR spectrometry and infrared spectrometry. The polymorphic form may also give rise the thermal behavior different from that of the amorphous material or another polymorphic form. Thermal behavior is measured in the laboratory by such techniques as capillary melting point, thermogfavimetric analysis (TGA) and differential scanning calorimetry (DSC) and can be used to distinguish some polymorphic forms from others.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for polymorphic forms of clopidogrel hydrobromide and processes for their preparation.

### SUMMARY OF THE INVENTION

In one aspect, the present invention provides a solid clopidogrel hydrobromide hydrate having a purity of at least about 99% by % area HPLC.

In another aspect the present invention is directed to the preparation of Form I characterized by a powder X-ray diffraction pattern having peaks at about 12.5, 15.8, 27.9 and 28.4±0.2 deg. 2-theta, comprising the steps of:
(a) providing a solution of (+)-clopidogrel or a salt thereof in a solvent;
(b) combining, while stirring, aqueous hydrobromic acid to crystallize the crystalline form; and
(c) recovering the crystalline form,

wherein the solvent is selected from the group consisting of C₃₋₆ alkyl esters and ketones, C₁₋₆ alcohols and C₃₋₆ ethers.

Preferred solvents include ethyl acetate, acetone, tetrahydrofuran, and iso-propanol. Most preferably, the solvent is ethyl acetate.

Preferably, the (+)-clopidogrel salt is (−)-camphor-10-sulfonate.

In another aspect, the process for preparing this crystalline form comprises exposing clopidogrel hydrobromide to air.

In another aspect, the present invention provides an anhydrous solid crystalline form of clopidogrel hydrobromide, having a melting point within the range of about 124° C. to about 138° C. Preferably, the melting range of this anhydrous solid crystalline form is of about 124° C. to about 128° C.

In another aspect, the present invention provides a solid crystalline form of clopidogrel hydrobromide, denominated Form II, having a melting point within the range of about 124° C. to about 138° C., and characterized by a powder X-ray diffraction pattern having peaks at about 9.6, 10.5, 14.3, 16.2 and 23.1±0.2 deg. 2-theta. Preferably, the melting range of Form II is of about 124° C. to about 128° C.

Another aspect of the invention is directed towards the preparation of this Form II, comprising the steps of:
(a) preparing a solution of clopidogrel hydrobromide in methyl acetate at a reflux temperature;
(b) crystallizing the crystalline form; and
(c) recovering the crystalline form.

In another aspect, the present invention provides a solid crystalline form of clopidogrel hydrobromide, denominated Form III, characterized by a powder X-ray diffraction pattern having peaks at about 7.5, 8.4, 19.5 and 24.0±0.2 deg. 2-theta.

In another aspect of the invention is directed towards the preparation of Form III, comprising the steps of:
(a) maintaining a heterogeneous mixture of clopidogrel hydrobromide Form I in 2-butanol for at least about 24 hours; and
(b) recovering the crystalline form.

In another aspect, the process for preparing Form III comprises the steps of:
(a) preparing a solution of clopidogrel hydrobromide in a solvent at a temperature of about 50° C. to about 85° C.;
(b) crystallizing the crystalline form; and
(c) recovering the crystalline form,

wherein the solvent is 2-butanol or dioxane.

Preferably, the heterogeneous mixture in step (a) is maintained while stirring it.

In another aspect, the present invention provides a solid crystalline form of clopidogrel hydrobromide, denominated Form IV, characterized by a powder X-ray diffraction pattern having peaks at about 20.7, 22.1, 23.0 and 25.1±0.2 deg. 2-theta.

In another aspect of the invention is directed towards the preparation of Form IV, comprising the steps of:
(a) preparing a solution of clopidogrel hydrobromide in a solvent at a temperature of at least about 50° C.;
(b) crystallizing the crystalline form; and
(c) recovering the crystalline form,

wherein the solvent is selected from the group consisting of acetone and mixtures of propylene glycol methyl ether, n-propanol or ethanol with heptane.

In another aspect, the present invention provides a solid crystalline form of clopidogrel hydrobromide, denominated Form V, characterized by a powder X-ray diffraction pattern having peaks at about 7.5, 8.8, 16.6 and 22.9±0.2 deg. 2-theta.

Another aspect of the invention is directed towards the preparation of a Form V comprising the steps of:
(a) preparing, at a temperature of at least about 50° C., a solution of clopidogrel hydrobromide in tetrahydrofuran;
(b) crystallizing the crystalline form;
(c) recovering the crystalline form.

In one aspect, the present invention provides a solid crystalline form of clopidogrel hydrobromide, denominated Form VI, characterized by a powder X-ray diffraction pattern having peaks at about 10.8, 21.5, 22.3 and 23.4±0.2 deg. 2-theta.

Another aspect of the invention is directed towards the preparation of Form VI, comprising the steps of:
(a) preparing, at a temperature of at least about 50° C., a solution of clopidogrel hydrobromide in dimethylcarbonate;
(b) crystallizing the crystalline form; and
(c) recovering the crystalline form.

In one aspect, the present invention provides a solid crystalline form of clopidogrel hydrobromide, denominated Form VII, characterized by a powder X-ray diffraction pattern having peaks at about 8.2, 9.0, 18.5 and 23.3±0.2 deg. 2-theta.

Another aspect of the invention is directed towards the preparation of Form VII, comprising the steps of:
(a) preparing, at a temperature of at least about 50° C., a solution of clopidogrel hydrobromide in ethyl acetate;
(b) crystallizing the crystalline form; and
(c) recovering the crystalline form.

In another embodiment, the process for preparing Form VII comprises the steps of:
(a) contacting clopidogrel hydrobromide with acetonitrile vapor to obtain the crystalline form; and
(b) recovering the crystalline form.

Another aspect of the invention is directed towards the preparation of Form VII, comprising the steps of:
(a) maintaining a heterogeneous mixture of clopidogrel hydrobromide in ethyl acetate and water;
(b) removing the ethyl acetate and water; and
(c) recovering the crystalline form.

In one aspect, the present invention provides a solid crystalline form of clopidogrel hydrobromide, denominated Form VIII, characterized by a powder X-ray diffraction pattern having peaks at about 10.4, 20.5, 22.8, 25.7 and 26.6±0.2 deg. 2-theta.

Another aspect of the invention is directed towards the preparation of Form VIII, comprising the steps of:
(a) maintaining a heterogeneous mixture of clopidogrel hydrobromide in a solvent; and
(b) recovering the crystalline form,

wherein the solvent is chlorobenzene or dichlorobenzene.

In one aspect, the present invention provides a solid crystalline form of clopidogrel hydrobromide, denominated Form IX, characterized by a powder X-ray diffraction pattern having peaks at about 7.9, 19.4, 19.8 and 24.0±0.2 deg. 2-theta.

Another aspect of the invention is directed towards the preparation of Form IX. In one embodiment, the process for preparing the crystalline form comprises the steps of:
(a) preparing a solution of clopidogrel hydrobromide in dioxane at a temperature of at least about 50° C.;
(b) crystallizing the crystalline form; and
(c) recovering the crystalline form.

In one aspect, the present invention provides a solid crystalline form of clopidogrel hydrobromide, denominated Form X, characterized by a powder X-ray diffraction pattern having peaks at about 9.7, 16.9, 17.2 and 19.5±0.2 deg. 2-theta.

Another aspect of the invention is directed towards the preparation of Form X.

In one embodiment, the process for preparing the crystalline form comprises the steps of:
(a) maintaining a heterogeneous mixture of clopidogrel hydrobromide in isopropanol; and
(b) recovering the crystalline form.

Another aspect of the present invention is directed towards pharmaceutical compositions comprising the hydrobromide salts of Forms II, III, IV, V, VI, VII, VIII, IX, X, and mixtures thereof.

Yet another aspect of the present invention is directed towards a method of reducing the occurrence of blood clots by administering the pharmaceutical compositions to a mammal in need thereof.

### BRIEF DESCRIPTION OF THE FIGURES

FIG. 1 is an X-Ray powder diffractogram of clopidogrel hydrobromide Form I.

FIG. 2 is an X-Ray powder diffractogram of clopidogrel hydrobromide Form II.

FIG. 3 is an X-Ray powder diffractogram of clopidogrel hydrobromide Form III.

FIG. 4 is an X-Ray powder diffractogram of clopidogrel hydrobromide Form IV.

FIG. 5 is an X-Ray powder diffractogram of clopidogrel hydrobromide Form V.

FIG. 6 is an X-Ray powder diffractogram of clopidogrel hydrobromide Form VI.

FIG. 7 is an X-Ray powder diffractogram of clopidogrel hydrobromide Form VII.

FIG. 8 is an X-Ray powder diffractogram of clopidogrel hydrobromide Form VIII.

FIG. 9 is an X-Ray powder diffractogram of clopidogrel hydrobromide Form IX.

FIG. 10 is an X-Ray powder diffractogram of clopidogrel hydrobromide Form X.

### DETAILED DESCRIPTION OF THE INVENTION

The solid crystalline forms of clopidogrel hydrobromide described in the present invention are (+)-Clopidogrel hydrobromide solid crystalline forms.

The present invention provides a crystalline clopidogrel hydrobromide hydrate having a purity of at least about 99% by % area HPLC. The major impurities are α-(2-chlorophenyl)-4,5,6,7-tetrahydrotieno[3,2-c]pyridyl-5-acetic acid and (−) clopidogrel.

This pure crystalline form exhibits only minor hygroscopicity, and ideal for formulation. This crystalline form of clopidogrel hydrobromide may contain particles having a particle size up to about 300 microns.

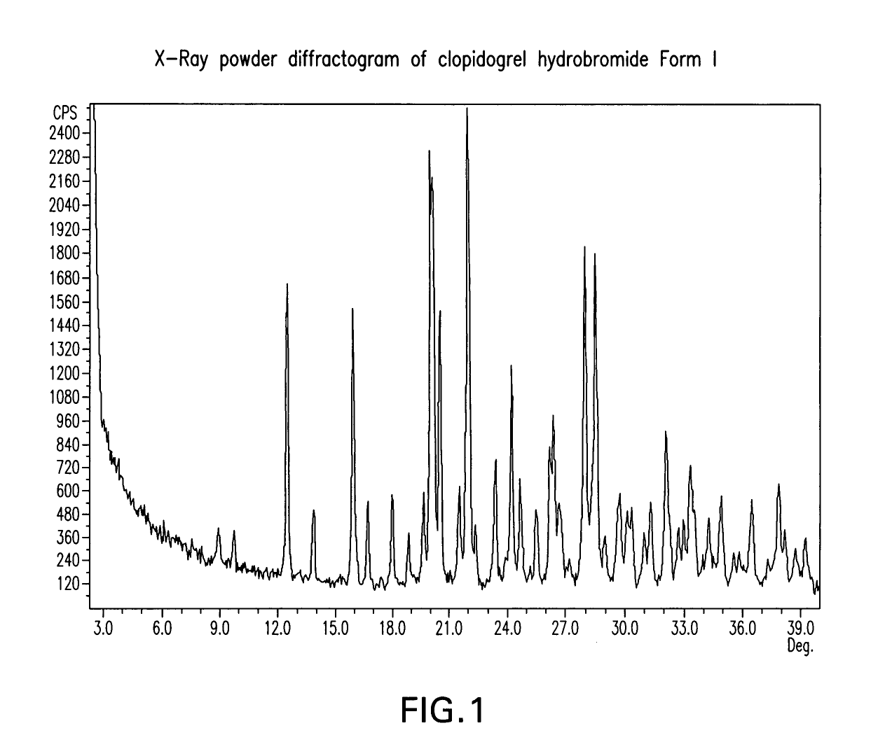

The present invention also provides a process for preparing clopidogrel hydrobromide Form I, characterized by a powder X-ray diffraction pattern having peaks at about 12.5, 15.8, 27.9 and 28.4±0.2 deg. 2-theta (substantially as depicted in FIG. 1). In one embodiment, the process for preparing the crystalline form includes combining aqueous hydrobromic acid with a solution of (+)-clopidogrel or a salt thereof in a solvent selected from the group consisting of C₃₋₆ alkyl esters and ketones, C₁₋₆ alcohols and C₃₋₆ ethers. Preferred solvents include ethyl acetate, acetone, tetrahydrofuran, isopropanol and mixtures thereof, to crystallize Form I. Most preferably, the solvent is ethyl acetate.

The (+)-clopidogrel salt is preferably (−)-camphor-10-sulfonate.

The acid may be added dropwise or in one or separate portions to the solution. The solution may be cooled to further accelerate the precipitation process. In one embodiment the solution is cooled at about room temperature or lower, and is stirred during the cooling time. The solution may also be seeded to accelerate the precipitation process.

In a preferred embodiment, the solution is heated at a temperature of about 30° C. to about 60° C. before addition of the hydrobromic acid, preferably at about 45° C. The hydrobromic acid is preferably added dropwise to the solution, more preferably with stirring. Preferably, the crystalline form is crystallized by cooling the solution after seeding. More preferably, the solution is cooled at about 0° C. to about 10° C., most preferably about 5° C. The solution is preferably stirred during cooling.

The crystalline form may be recovered by any conventional technique, such as filtration, and may be dried. Drying may be carried out under reduced pressure and/or elevated temperature. In one embodiment, the filtered solid is recovered by heating under a pressure of less than about 100 mm Hg. Preferably, the recovered crystalline form is heated at a temperature of about 30° C. to about 60° C.

When the (+)-clopidogrel salt is used, a pure crystalline Form I is obtained. Preferably, the purity of the crystalline Form I obtained is at least about 99% area by HPLC.

Form I may also be prepared by exposing a hygroscopic crystalline clopidogrel hydrobromide to air, preferably for at least about 24 hours. In a preferred embodiment, the clopidogrel hydrobromide exposed is Form II, discussed below.

The present invention also provides an anhydrous crystalline form of clopidogrel hydrobromide, having a melting point within the range of about 124° C. to about 138° C. Preferably, the melting range of this anhydrous solid crystalline form is of about 124° C. to about 128° C. This crystalline form of clopidogrel hydrobromide may contain particles having a particle size up to about 300 microns. Also, this solid crystalline form may be polymorphically pure, i.e. contain no more than about 10% of other forms, as measured by XRD.

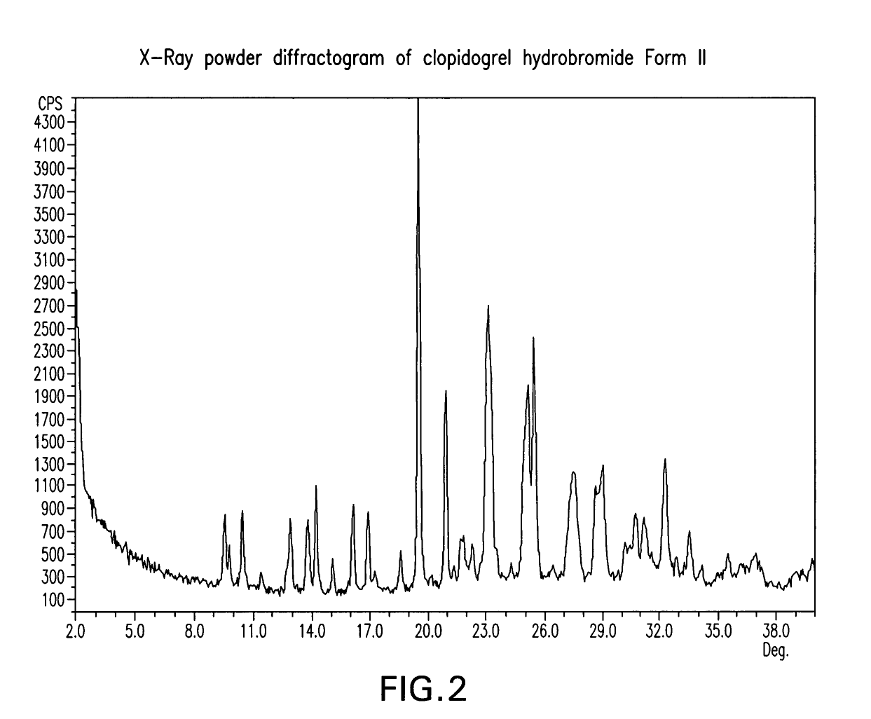

The present invention further provides a solid crystalline form of clopidogrel hydrobromide, denominated Form II, having a melting point within the range of about 124° C. to about 138° C., and characterized by a powder X-ray diffraction pattern having peaks at about 9.6, 10.5, 14.3, 16.2 and 23.1±0.2 deg. 2-theta. Preferably, the melting range of Form II is of about 124° C. to about 128° C. Clopidogrel hydrobromide Form II is anhydrous. Anhydrous forms generally contain less than about 2% water by weight. Form II may be further characterized by a powder X-Ray diffraction pattern with peaks at about 12.9, 13.8, 19.5, 20.9, 25.1 and 25.5±0.2 deg. 2-theta. A typical x-ray diffraction diagram for clopidogrel hydrobromide Form II is given in FIG. 2. Crystalline clopidogrel hydrobromide Form II of the present invention may contain particles having a particle size up to about 300 microns. Also, the solid crystalline Form II of the present invention may be polymorphically pure, i.e., contain no more than about 10% of other forms, as measured by XRD, such as Form I as area percentage XRD.

The present invention also provides a process for preparing clopidogrel hydrobromide Form II by crystallization from a solution of clopidogrel hydrobromide in methyl acetate. Preferably, the solution is heated, more preferably to reflux temperature, and cooled to induce crystallization. Preferably the solution is cooled to room temperature or lower.

Recovery and drying of the crystals may be carried out as described above.

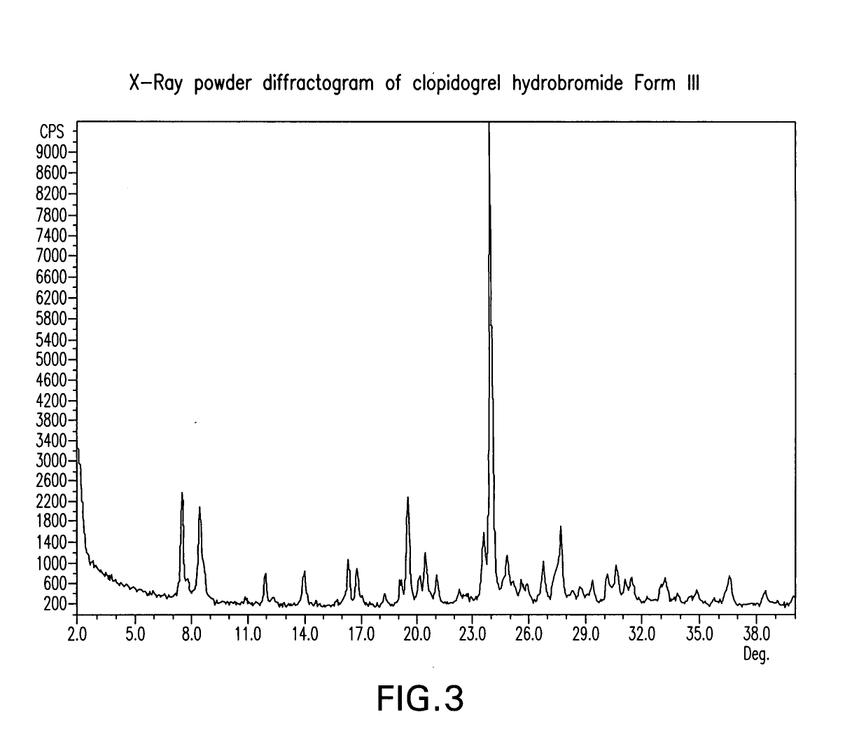

The present invention also provides a solid crystalline form of clopidogrel hydrobromide, denominated Form III, characterized by a powder X-ray diffraction pattern having peaks at 7.5, 8.4, 19.5 and 24.0±0.2 deg. 2-theta. Form III may be further characterized by a powder X-Ray diffraction pattern having peaks at about 11.9, 14.0, 16.3, 20.5, 26.8 and 27.7±0.2 deg.2-theta. A typical x-ray diffraction diagram for clopidogrel hydrobromide Form III is given in FIG. 3. Crystalline clopidogrel hydrobromide Form III of the present invention may contain particles having a particle size up to about 300 microns. The solid crystalline Form III of the present invention may be polymorphically pure, i.e., contain no more than about 10% of other forms, as measured by XRD, such as Form I or Form II.

The present invention also provides a process for preparing clopidogrel hydrobromide Form III by maintaining a heterogeneous mixture of clopidogrel hydrobromide in 2-butanol and recovering the crystalline form after the transition to Form III. Preferably the starting Form is Form I. Form III may also be prepared by crystallization from 2-butanol or dioxane.

In a preferred embodiment, when the solvent is dioxane, the ratio of dioxane to clopidogrel hydrobromide is greater than about 4 ml/g. Preferably the solution is heated at a temperature of about 50° C. to about 85° C. Preferably, the solution is prepared by dissolving clopidogrel HBr hydrate, so that there is water present in the reaction mixture. Crystallization may be induced by cooling after heating. Preferably, the solution is cooled to room temperature or lower.

Recovery and drying may be carried out as described above.

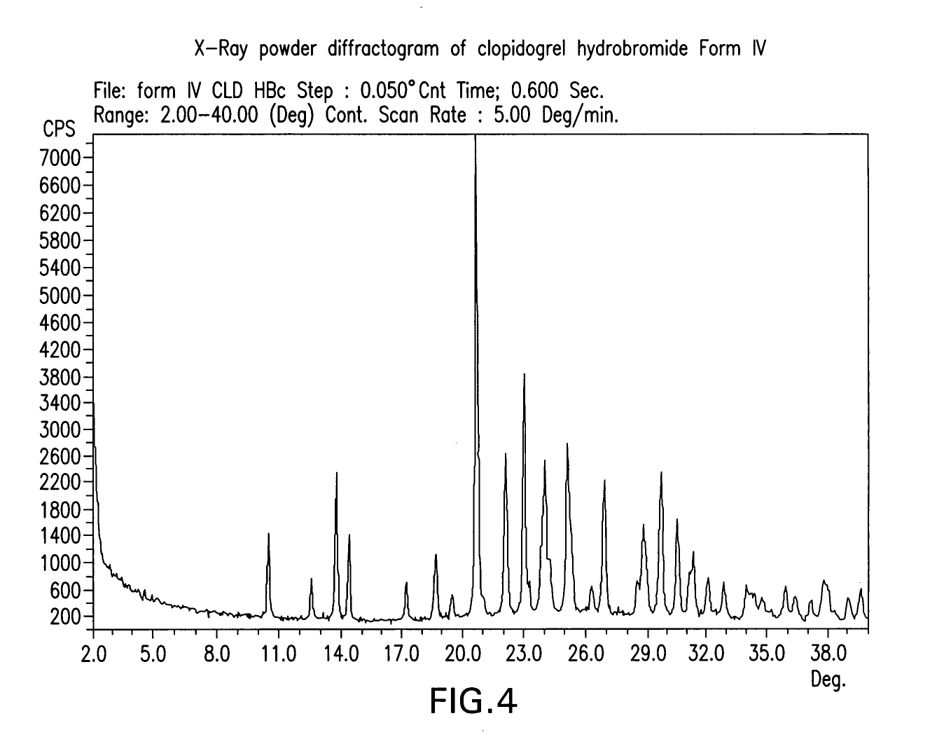

The present invention provides a solid crystalline form of clopidogrel hydrobromide, denominated Form IV, characterized by a powder X-ray diffraction pattern having peaks at about 20.7, 22.1, 23.0 and 25.1±0.2 deg. 2-theta. Form IV may be further characterized by a powder X-Ray diffraction pattern having peaks at about 10.5, 13.8, 26.9 and 29.7±0.2 deg. 2-theta. A typical x-ray diffraction diagram for clopidogrel hydrobromide Form IV is given in FIG. 4. Crystalline clopidogrel hydrobromide Form IV of the present invention may contain particles having a particle size up to about 300 microns. Also, the solid crystalline Form IV of the present invention may be polymorphically pure, i.e., contain no more than about 10% of other forms, as measured by XRD, such as Form I.

The present invention also provides a process for preparing clopidogrel hydrobromide Form IV by crystallization from solvent systems such as mixtures of propylene glycol methyl ether, n-propanol or ethanol with heptane. Form IV may also be prepared by crystallization from acetone. These processes are preferably performed by heating the solutions to a temperature of at least about 50° C., more preferably a temperature of about 50° C. to about reflux. Crystallization may be induced by cooling the heated solution. Preferably, the solution is cooled to room temperature or lower.

The crystals may be recovered and dried as above.

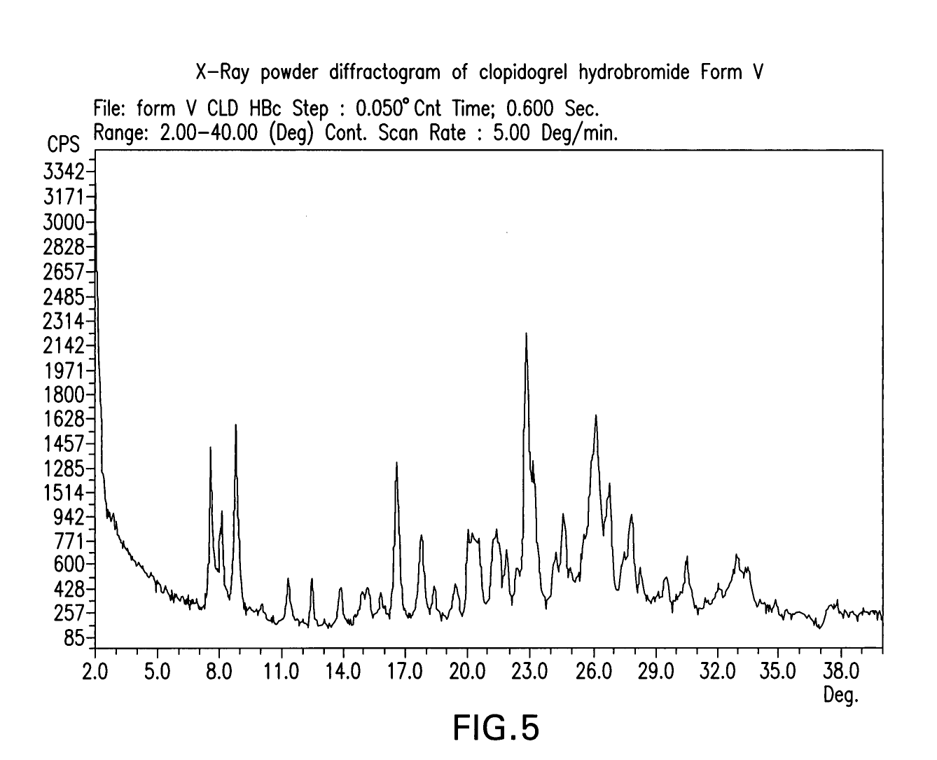

The present invention also provides a solid crystalline form of clopidogrel hydrobromide, denominated Form V, characterized by a powder X-ray diffraction pattern having peaks at about 7.5, 8.8, 16.6 and 22.9±0.2 deg. 2-theta. Form V may be further characterized by a powder X-Ray diffraction pattern having peak at about 26.2±0.2 deg. 2-theta. A typical x-ray diffraction diagram for clopidogrel hydrobromide Form V is given in FIG. 5. Crystalline clopidogrel hydrobromide Form V of the present invention may contain particles having a particle size up to about 300 microns. Also, the solid crystalline Form V of the present invention may be polymorphically pure, i.e., contain no more than about 10% of other forms, as measured by XRD, such as Form I.

The present invention also provides a process for the preparation of clopidogrel hydrobromide Form V by crystallization from tetrahydrofuran where the solution is heated to at least about 50° C., more preferably reflux temperature. Preferably the heated solution is cooled to room temperature or lower to induce crystallization.

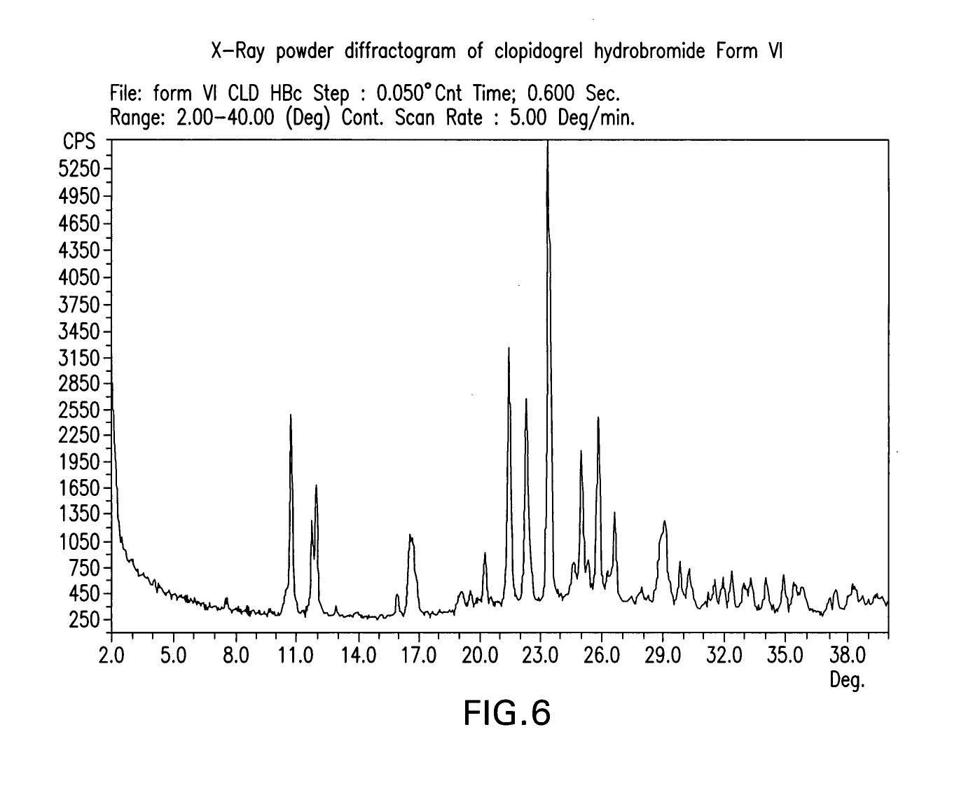

The present invention also provides a solid crystalline form of clopidogrel hydrobromide, denominated Form VI, characterized by a powder X-ray diffraction pattern having peaks at about 10.8, 21.5, 22.3 and 23.4±0.2 deg. 2-theta. Form VI may be further characterized by a powder X-Ray diffraction pattern having peaks at about 12.0 and 25.9±0.2 deg. 2-theta. A typical x-ray diffraction diagram for clopidogrel hydrobromide Form VI is given in FIG. 6. Crystalline clopidogrel hydrobromide Form VI of the present invention may contain particles having a particle size up to about 300 microns. Also, the solid crystalline Form VI of the present invention may be polymorphically pure, i.e., contain no more than about 10% of other forms, as measured by XRD, such as Form I.

The present invention also provides a process for preparing clopidogrel hydrobromide Form VI by crystallization from dimethylcarbonate. In a preferred embodiment, the solution is heated at a temperature of at least about 50° C., and crystallization is induced by cooling to room temperature or lower. Most preferably, the solution is heated to a temperature of about 85° C.

Recovery and drying may be carried out as described above.

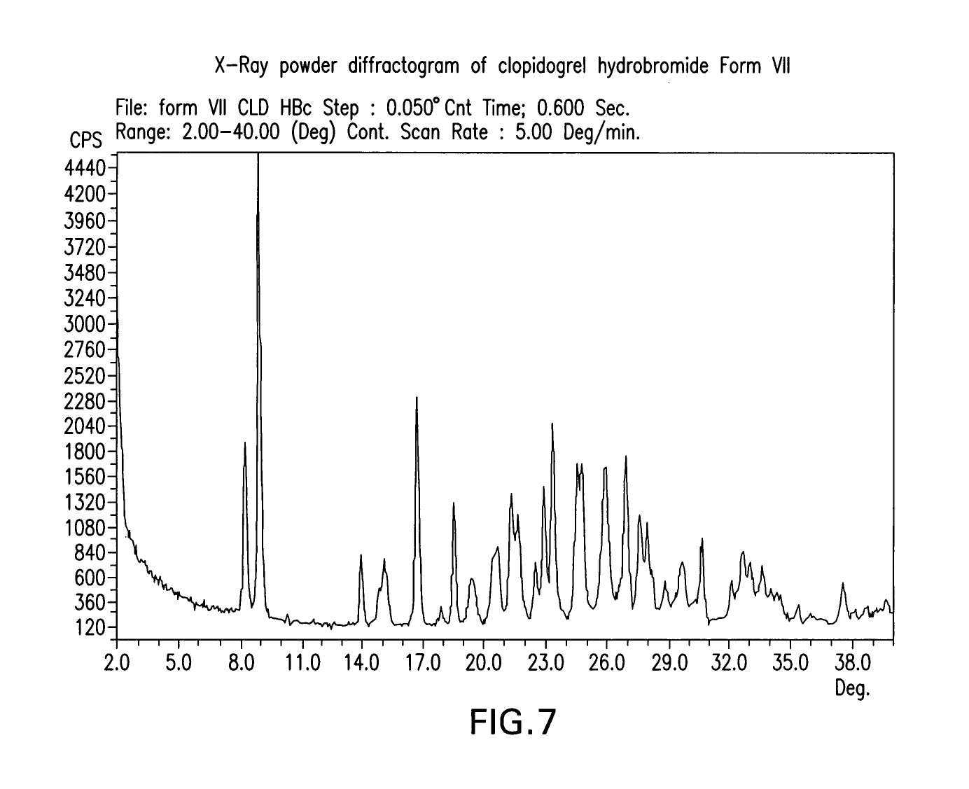

The present invention also provides a solid crystalline form of clopidogrel hydrobromide, denominated Form VII, characterized by a powder X-ray diffraction pattern having peaks at about 8.2, 9.0, 18.5 and 23.3±0.2 deg. 2-theta. Form VII may be further characterized by a powder X-Ray diffraction pattern having peaks at about 16.7 and 26.9±0.2 deg. 2-theta. A typical x-ray diffraction diagram for clopidogrel hydrobromide Form VII is given in FIG. 7. Crystalline clopidogrel hydrobromide Form VII of the present invention may contain particles having a particle size up to about 300 microns. Also, the solid crystalline Form VII of the present invention may be polymorphically pure, i.e., contain no more than about 10% of other forms, as measured by XRD, such as Form I.

The present invention also provides a process for preparing clopidogrel hydrobromide Form VII by crystallization from ethyl acetate, where the solution is heated to at temperature of least about 50° C., more preferably reflux temperature. Crystallization is preferably induced by cooling the heated solution to room temperature or lower.

Clopidogrel hydrobromide Form VII may also be prepared by contacting crystalline clopidogrel hydrobromide with acetonitrile vapor for a sufficient time to obtain transformation. Preferably the crystalline clopidogrel hydrobromide contacted is Form II. Contact for at least about 7 days is preferred.

The present invention also provides a process for preparing Form VII by maintaining a heterogeneous mixture (i.e. slurry) of crystalline clopidogrel hydrobromide in ethyl acetate and water for a sufficient time to obtain a transformation, and then removing the ethyl acetate and water to obtain Form VII. Preferably the crystalline clopidogrel hydrobromide is Form II and the amount of water is less than about 1% by volume. Removal of the ethyl acetate and water may be by filtration and drying.

When this process is performed without the drying step, a mixture of Forms VII and I is obtained.

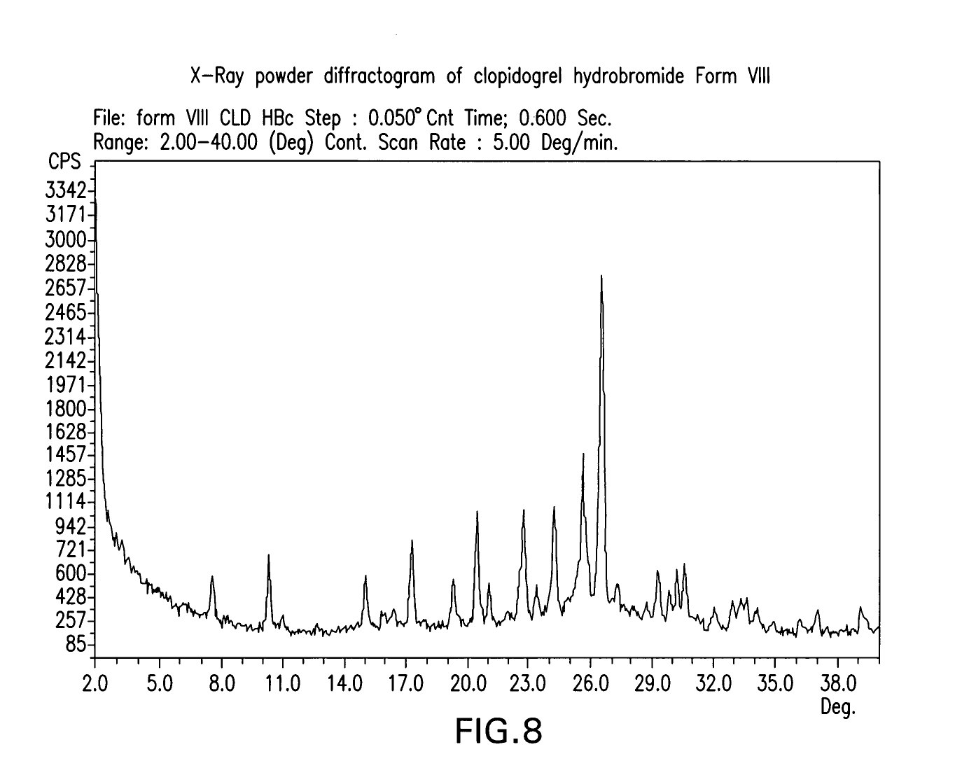

The present invention also provides a solid crystalline form of clopidogrel hydrobromide, denominated Form VIII, characterized by a powder X-ray diffraction pattern having peaks at about 10.4, 20.5, 22.8, 25.7 and 26.6±0.2 deg. 2-theta. Form VIII may be further characterized by a powder X-Ray diffraction pattern having peaks at about 7.5, 15.0, 17.3 and 24.3±0.2 deg. 2-theta. A typical x-ray diffraction diagram for clopidogrel hydrobromide Form VIII is given in FIG. 8. Crystalline clopidogrel hydrobromide Form VIII of the present invention may contain particles having a particle size up to about 300 microns. Also, the solid crystalline Form VIII of the present invention may be polymorphically pure, i.e., contain no more than about 10% of other forms, as measured by XRD, such as Form I.

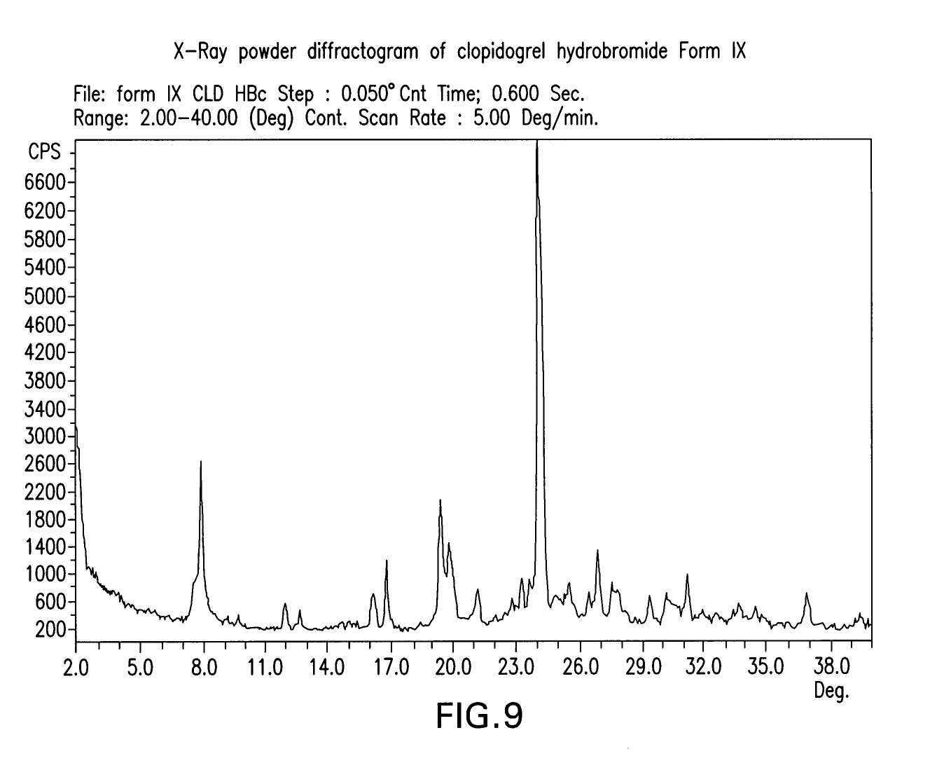

The present invention also provides a solid crystalline form of clopidogrel hydrobromide, denominated Form IX, characterized by a powder X-ray diffraction pattern having peaks at about 7.9, 19.4, 19.8 and 24.0±0.2 deg. 2-theta. Form IX may be further characterized by a powder X-Ray diffraction pattern having peaks at about 16.1 and 16.7±0.2 deg. 2-theta. A typical x-ray diffraction diagram for clopidogrel hydrobromide Form IX is given in FIG. 9. Crystalline clopidogrel hydrobromide Form IX of the present invention may contain particles having a particle size up to about 300 microns. Also, the solid crystalline Form IX of the present invention may be polymorphically pure, i.e., contain no more than about 10% of other forms, as measured by XRD, such as Form I.

The present invention also provides a process for preparing clopidogrel hydrobromide Form IX by crystallization from dioxane. Preferably, anhydrous clopidogrel HBr is dissolved in dioxane, so that there is no water present in the reaction mixture. Preferably, the clopidogrel HBr dissolved in dioxane is Form II. In a preferred embodiment, the ratio of dioxane to clopidogrel hydrobromide is less than about 4 ml/g. The solution may be heated at a temperature of about 50° C. to about 85° C., followed by cooling to room temperature or lower to induce crystallization.

Recovery and drying may be carried out as described above.

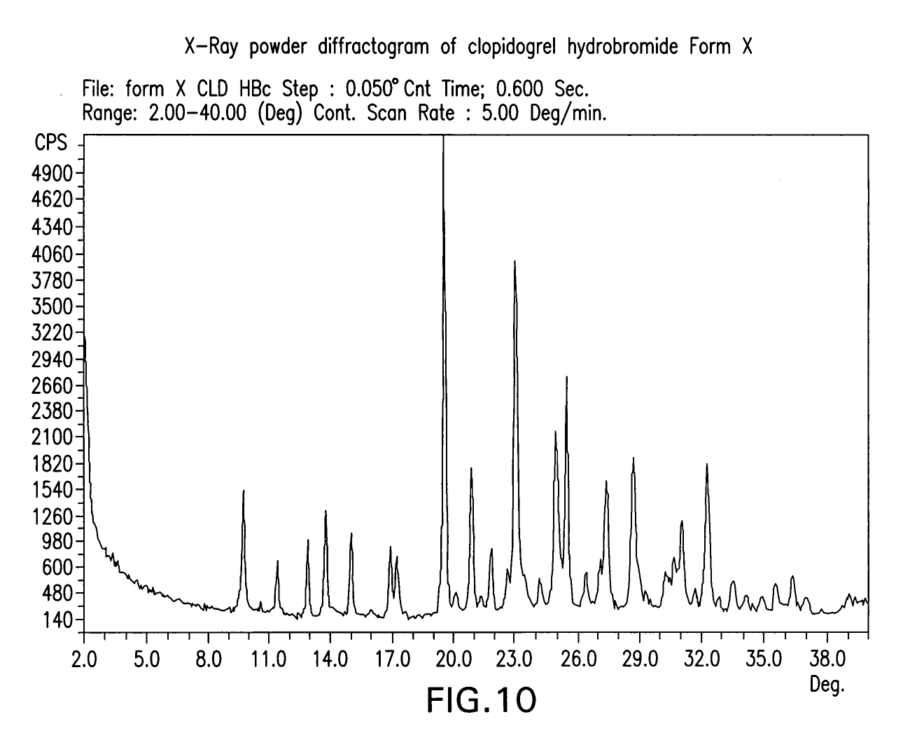

The present invention also provides a solid crystalline form of clopidogrel hydrobromide, denominated Form X, characterized by a powder X-ray diffraction pattern having peaks at about 9.7, 16.9, 17.2 and 19.5±0.2 deg. 2-theta. Form X may be further characterized by a powder X-Ray diffraction pattern having peaks at about 11.4, 12.9, 13.8, 23.0, 24.9 and 25.5±0.2 deg. 2-theta. A typical x-ray diffraction diagram for clopidogrel hydrobromide Form X is given in FIG. 10. Crystalline clopidogrel hydrobromide Form X of the present invention may contain particles having a particle size up to about 300 microns. Also, the solid crystalline Form X of the present invention may be polymorphically pure, i.e., contain no more than about 10% of other forms, as measured by XRD, such as Form I.

The present invention also provides a process for preparing clopidogrel hydrobromide Form X by maintaining a heterogeneous mixture of crystalline clopidogrel hydrobromide in isopropanol for a sufficient time to obtain a transformation. Preferably the crystalline clopidogrel hydrobromide is Form I.

Form X may be recovered and dried as described above.

One of skill in the art would appreciate that as the slurry (a heterogeneous mixture) is allowed to last for a sufficient time to obtain a particular polymorphic form, the slurry may dry up due to for example evaporation of the solvents. As the examples illustrate, additional amounts of a solvent may be added (same or different solvent), preferably followed by stirring, to obtain a slurry.

Some processes of the present invention involve crystallization out of a particular solvent. One skilled in the art would appreciate that the conditions concerning crystallization can be modified without affecting the form of the polymorph obtained. For example, when mixing clopidogrel hydrobromide in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization. The conditions may also be changed to induce precipitation. A preferred way of inducing precipitation is to reduce the solubility of the solute. The solubility of the solute may be reduced, for example, by cooling the solvent. For example, as illustrated in the experiments, the solution was cooled to room temperature, followed by an ice bath to crystallize the crystalline forms.

The clopidogrel hydrobromide may be used as an intermediate in the preparation of clopidogrel bisulfate. Clopidogrel bromide may either be converted directly the bisulfate by addition of hydrosulfuric acid, or first be converted to clopidogrel base by reaction with a base, followed by conversion of the free base to the bisulfate salt. Examples of preparation of the bisulfate salt from clopidogrel base are provided in U.S. Pat. No. 6,767,913, incorporated herein by reference.

Pharmaceutical compositions of the present invention contain crystalline forms of clopidogrel hydrobromide, such as one of those disclosed herein, optionally in mixture with other form(s) of clopidogrel hydrobromide. In addition to the active ingredient(s), the pharmaceutical compositions of the present invention may contain one or more excipients. Excipients are added to the composition for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, clopidogrel and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxyl toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the composition scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular composition challenges of direct compression tableting.

A capsule filling of the present invention may comprise any of the aforementioned blends and granulates that were described with reference to tableting, however, they are not subjected to a final tableting step.

The dosage of PLAVIX® may be used as guidance. PLAVIX® is administered orally. The recommended oral dose of PLAVIX® is 75 mg once daily.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Instrumentation

X-ray powder diffraction data were obtained by methods known in the art using a SCINTAG powder X-ray diffractometer model X'TRA equipped with a solid state detector. Copper radiation of 1.5418 Å was used. A round aluminum sample holder with round zero background quartz plate, with cavity of 25(diameter)*0.5(depth) mm. The obtained characteristic peaks were in the range of 2-40 degrees two theta.

For measuring particle size the following main methods are employed: sieves, sedimentation, electrozone sensing (coulter counter), microscopy, Low Angle Laser Light Scattering (LALLS).

### Impurity Profile Determination

| | |
|---|---|
| Column & Packing: | Phenyl 5 μm 4.6 * 250 mm |
| Eluent: | 5 g Dodecyl sulfate sodium salt dissolve in 500 ml |
| | water, adjust to pH 3.0 with H₃PO₄ |
| | 420 ml Acetonitrile, 80 ml Methanol |
| Flow Rate: | 1.3 ml/min |
| Detector: | 220 nm |
| Sample Volume: | 10 μμl |
| Diluent: | eluent |
| Sample preparation: | 0.5 mg/ml for impurity profile determination |
| | 0.05 mg/ml for assay determination |

### Procedures:

For preparation (+)-clopidogrel hydrobromide from (+)-clopidogrel and 48% aqueous hydrobromic acid, solvents, such as ethyl acetate, acetone, tetrahydrofuran and iso-propyl alcohol, were used. The obtained salts may be monohydrates.

### Form I

Table 1 below summarizes purity of (+)-clopidogrel hydrobromide for examples 1-4 (HPLC analysis) detailed below.

TABLE 1

| Example | Area % - Impurity profile | Assay % |
|---|---|---|
| 1 | 100 | 98.92 |
| 2 | 100 | 98.31 |
| 3 | 99.95 | 99.21 |
| 4 | 100 | 100 |

### Example 1

A solution of (+)-clopidogrel (10.0 g) in 90 ml of ethyl acetate was vigorously stirred with 48% aqueous hydrobromic acid (3.6 ml) at room temperature overnight. The solid was filtered and washed with ethyl acetate giving, after drying under vacuum at 40° C. for 6 hours, 10.2 g (79%) of (+)-clopidogrel hydrobromide form I. The procedure was repeated twice. KF values were 4.3%, mp was 113° C. and 105° C.

### Example 2

A solution of (+)-clopidogrel (6.0 g) in 18 ml of acetone was vigorously stirred with 48% aqueous hydrobromic acid (2.2 ml) at room temperature overnight. The solid was filtered and washed with acetone giving, after drying under vacuum at 40° C. for 6 hours, 5.5 g (70%) of (+)-clopidogrel hydrobromide form I. KF value was 4.3% and mp was 107° C.

### Example 3

A solution of (+)-clopidogrel (6.0 g) in 30 ml of tetrahydrofuran was vigorously stirred with 48% aqueous hydrobromic acid (2.2 ml) at room temperature overnight. The solid was filtered and washed with tetrahydrofuran giving, after drying under vacuum at 40° C. for 6 hours, 6.2 g (80%) of (+)-clopidogrel hydrobromide form I. KF value was 4.4% and mp was 107° C.

### Example 4

A solution of (+)-clopidogrel (6.0 g) in 30 ml of iso-propanol was vigorously stirred with 48% aqueous hydrobromic acid (2.2 ml) at room temperature overnight. The solid was filtered and washed with iso-propanol giving, after drying under vacuum at 40° C. for 6 hours, 5.5 g (70%) of (+)-clopidogrel hydrobromide form I. KF value was 4.6% and the mp was 107° C.

### Example 5

Clopidogrel hydrobromide (form II) (1.0 g) was kept in opened beaker at room temperature for 4 days giving clopidogrel hydrobromide form I. The mp was 103° C.

### Example 6

A solution of (+) clopidogel (prepared from 1.6 kg of (+)clopidogrel (−) camphor-10-sulfonate) in 9.6 L of ethyl acetate was heated to 30° C. Aqueous hydrobromic acid (48%, 481 g) was added dropwise to the solution for 1 hour with vigorous stirring. The mixture was cooled to 20° C. for 6 hours with vigorous stirring. The solid was filtered, washed with ethyl acetate, and gave after drying (45° C., under vacuum overnight) 1.15 kg of (+) clopidogrel hydrobromide (form I).

KF value: 4.3%, m.p. 104° C. Chromatographic purity (by HPLC): 100%. Assay (by HPLC): 99.7%.

### Example 7

A solution of (+) clopidogrel base (prepared from 1.12 kg (+) clopidogrel (−) camphor-10-sulfonate) in 6.7 L ethyl acetate was heated to 45° C. Aqueous hydrobromic acid (48%, 353 g) was added dropwise for 2 hours with vigorous stirring. The mixture was seeded with 1 g of (+) clopidogrel hydrobromide (form I) and cooled to 5° C. for 8 hours with vigorous stirring. The solid was filtered, washed with ethyl acetate, and gave after drying (45° C., under vacuum overnight) 0.75 kg of (+) clopidogrel hydrobromide (form I).

KF value: 4.2%, m.p. 107.5° C. Chromatographic purity (by HPLC): 99.68%. Assay (by HPLC): 98.7%.

### Form II

### Example 8

(+)Clopidogrel hydrobromide (form I) (2.5 g) was dissolved in 20 ml of methyl acetate by heating at reflux. The mixture was cooled to room temperature and then on ice bath for additional two hours. The solid was filtered and gave after drying overnight under vacuum at 50° C. (+)clopidogrel hydrobromide (form II). The melting range for the obtained product was 124-128° C.

### Example 9

### (+)Clopidogrel hydrobromide (form I) (3 g) was slurried with 15 ml of tert-butanol at room temperature for 24 hours. The solid was filtered, washed with the solvent giving clopidogrel hydrobromide (form II). The melting range for the obtained product was 124-128° C.

### Form III

### Example 10

(+) Clopidogrel hydrobromide (form I) (3.0 g) was slurried with 33 ml of 2-butanol at room temperature for 24 hours. The solid was filtered, washed with 2-butanol and dried in vacuum at 40° C. overnight (wet form III).

### Example 11

(+)Clopidogrel hydrobromide (form I) (2.5 g) was dissolved in 12 ml of 2-butanol by heating at 85° C. The mixture was cooled to room temperature and then on ice bath for additional two hours. The solid was filtered and gave after drying overnight under vacuum at 50° C. (+)clopidogrel hydrobromide (wet form III, dry form III).

### Example 12

(+)Clopidogrel hydrobromide (form I) (2.5 g) was dissolved in 20 ml of dioxane by heating at 85° C. The mixture was cooled to room temperature and then on ice bath for additional two hours. The solid was filtered and gave after drying overnight under vacuum at 50° C. (+)clopidogrel hydrobromide (wet form III).

### Example 13

(+)Clopidogrel hydrobromide (form II) (1.0 g) was dissolved in 10 ml of 2-butanol by heating at 85° C. The mixture was cooled to room temperature and then on ice bath for additional two hours. The solid was filtered and gave after drying overnight under vacuum at 50° C. (+)clopidogrel hydrobromide (wet form III).

### Form IV

### Example 14-17

(+)Clopidogrel hydrobromide (form II) (1.0 g) was dissolved in solvents (see Table 2 below) by heating at 85° C. The mixture was cooled to room temperature and then on ice bath for additional two hours. The solid was filtered and gave after drying overnight under vacuum at 50° C. (+)clopidogrel hydrobromide (form IV).

TABLE 2

| example | solvents in ml |
|---|---|
| 14 | PGME*-heptane: 10-8 |
| 15 | n-propanol-heptane: 8-14 |
| 16 | ethanol-heptane: 10-22.5 |
| 17 | acetone, 10 volumes |

*PGME = propyleneglycol methyl ether, or 1-methoxy-2-propanol

### Form V

### Example 18

(+)Clopidogrel hydrobromide (form II) (1.0 g) was dissolved in 20 ml of tetrahydrofuran by heating at reflux. The mixture was cooled to room temperature and then on ice bath for additional two hours. The solid was filtered and gave after drying overnight under vacuum at 50° C. (+)clopidogrel hydrobromide (wet form V, dry form II+VI).

### Form VI

### Example 19

(+)Clopidogrel hydrobromide (form II) (1.0 g) was dissolved in 10 ml of dimethylcarbonate by heating at 85° C. The mixture was cooled to room temperature and then on ice bath for additional two hours. The solid was filtered and gave after drying overnight under vacuum at 50° C. (+)clopidogrel hydrobromide (wet form VI).

### Form VII

### Example 20

(+)Clopidogrel hydrobromide (form II) (1.0 g) was dissolved in 60 ml of ethyl acetate by heating at reflux. The mixture was cooled to room temperature and then on ice bath for additional two hours. The solid was filtered and gave after drying overnight under vacuum at 50° C. (+)clopidogrel hydrobromide (wet form VII, dry form VII).

### Example 21

(+)Clopidogrel hydrobromide (form II) (1.0 g) was slurried with 3 ml of ethyl acetate containing 0.025 ml of water. After (24) hours 8 ml of ethyl acetate was added and the suspension was slurried for additional 12 hours at room temperature. The solid was filtered and gave after drying overnight under vacuum at 50° C. (+)clopidogrel hydrobromide (wet form I+VII).

### Example 22

(+)Clopidogrel hydrobromide (Form I, 0.5 g) was put in a glass beaker. The beaker was put into a bigger closed vessel (the vessel volume 125 ml), containing 20 ml of acetonitrile. After 3 weeks crystals of (+)clopidogrel hydrobromide (form VII) were obtained.

### Form VIII

### Example 23

(+)Clopidogrel hydrobromide (form II) (3.0 g) was slurried with 24 ml of chlorobenzene for 24 hours at room temperature. The solid was filtered and gave after drying overnight under vacuum at 50° C. (+)clopidogrel hydrobromide (wet form VIII).

### Example 24

(+)Clopidogrel hydrobromide (form II) (2.0 g) was slurried with 6 ml of dichlorobenzene for 24 hours at room temperature. The solid was filtered and gave after drying overnight under vacuum at 50° C. (+)clopidogrel hydrobromide (wet form VIII, dry form VIII).

### Form IX

### Example 25

(+)Clopidogrel hydrobromide (form II) (1.0 g) was dissolved in 10 ml of dioxane by heating at 85° C. The mixture was cooled to room temperature and then on ice bath for additional two hours. The solid was filtered and gave after drying overnight under vacuum at 50° C. (+)clopidogrel hydrobromide (wet form IX).

### Form X

### Example 26

(+)Clopidogrel hydrobromide (form I) (3 g) was slurried with 12 ml of iso-propyl alcohol at room temperature for 24 hours. The solid was filtered and gave after drying overnight under vacuum at 50° C. (+)clopidogrel hydrobromide (wet form X, dry form X).

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used for guidance.

What is claimed is:

1. An anhydrous solid crystalline form of clopidogrel hydrobromide, having a melting point within the range of about 124° C. to about 138° C.

2. The anhydrous solid crystalline form of claim 1, having a melting range of about 124° C. to about 128° C.
